(19) United States
(12) Patent Application Publication
(10) Pub. No.: US 2010/0036432 A1
(43) Pub. Date: Feb. 11, 2010

(54) TWIST OFF REDUCTION SCREW

(75) Inventor: Kameron Scott Ely, Cedar Park, TX (US)

Correspondence Address: SPRINKLE IP LAW GROUP, 1301 W. 25TH STREET, SUITE 408, AUSTIN, TX, 78705, US

(73) Assignee: Abbott Spine Inc., Austin, TX (US)

(21) Appl. No.: 12/186,470

(22) Filed: Aug. 5, 2008

(51) Int. Cl. A61B 17/04

(52) U.S. Cl. 606/301; 606/305; 606/308; 606/302

(57) ABSTRACT

Methods and systems for reducing spinal stabilization rods. In one embodiment, a method can include attaching a collar to a boney structure. The collar can include a body and an extension, each including arms further defining threaded slots. A separation member can couple the body and the extension to each other. The method can include reducing the rod using the collar and separating the extension from the body by applying a torque to the extension. The separation of the extension from the body can be accomplished with one substantially continuous motion and can leave tissues adjacent to the collar substantially undisturbed. The arms of the extension can be prevented from splaying, thereby mitigating a moment during the reduction of the spinal stabilization rod through the extension. Thus, the separation member can transmit tensile forces and certain amounts of torques between the body and the extension.

### TECHNICAL FIELD

This disclosure relates generally to spinal stabilization systems, and more particularly to spinal implants for attaching spinal stabilization rods which may be proud of the spinal implants by some distance to boney structures associated with human spines.

### BACKGROUND

The human spine consists of segments known as vertebrae separated by intervertebral disks and held together by various ligaments. There are 24 movable vertebrae—7 cervical, 12 thoracic, and 5 lumbar. Each of the movable vertebra has a somewhat cylindrical bony body (often referred to as the centrum), a number of winglike projections, and a bony arch. The bodies of the vertebrae form the supporting column of the skeleton. The arches of the vertebrae are positioned so that the spaces they enclose form a curvilinear passage which is often referred to as the vertebral canal. The vertebral canal houses and protects the spinal cord (which includes bundles of sensory and motor nerves for sensing conditions in or affecting the body and commanding movements of various muscles). Within the vertebral canal, spinal fluid can circulate to cushion the spinal cord and carry immunological cells to it, thereby protecting the sensory and motors nerves therein from mechanical damage and disease. Ligaments and muscles are attached to various projections of the vertebrae such as the superior-inferior, transverse, and spinal processes. Other projections, such as vertebral facets, join adjacent vertebrae to each other, in conjunction with various attached muscles, tendons, etc. while still allowing the vertebrae to move relative to each other.

Spines may be subject to abnormal curvature, injury, infections, tumor formation, arthritic disorders, punctures of the intervertebral disks, slippage of the intervertebral disks from between the vertebrae, or combinations thereof. Injury or illness, such as spinal stenosis and prolapsed disks may result in intervertebral disks having a reduced disk height, which may lead to pain, loss of functionality, reduced range of motion, disfigurement, and the like. Scoliosis is one relatively common disease which affects the spinal column. It involves moderate to severe lateral curvature of the spine and, if not treated, may lead to serious deformities later in life. Such deformities can cause discomfort and pain to the person affected by the deformity. In some cases, various deformities can interfere with normal bodily functions. For instance, some spinal deformities can cause the affected person's rib cage to interfere with movements of the respiratory diaphragm, thereby making respiration difficult. Additionally, some spinal deformities noticeably alter the posture, gate, appearance, etc. of the affected person, thereby causing both discomfort and embarrassment to those so affected. One treatment involves surgically implanting devices to correct such deformities, to prevent further degradation, and to mitigate symptoms associated with the conditions which may be affecting the spine.

Modern spine surgery often involves spinal stabilization through the use of spinal implants or stabilization systems to correct or treat various spine disorders and/or to support the spine. Spinal implants may help, for example, to stabilize the spine, correct deformities of the spine, facilitate fusion of vertebrae, or treat spinal fractures and other spinal injuries. Spinal implants can alleviate much of the discomfort, pain, physiological difficulties, embarrassment, etc. that may be associated with spinal deformities, diseases, injury, etc.

Spinal stabilization systems typically include corrective spinal instrumentation that is attached to selected vertebra of the spine by bone anchors, screws, hooks, clamps, and other implants hereinafter referred to as “bone anchors.” Some corrective spinal instrumentation includes spinal stabilization rods, spinal stabilization plates that are generally parallel to the patient's back, or combinations thereof. In some situations, corrective spinal instrumentation may also include superior-inferior connecting rods that extend between bone anchors (or other attachment instrumentation) attached to various vertebrae along the affected portion of the spine and, in some situations, adjacent vertebrae or adjacent boney structures (for instance, the occipital bone of the cranium or the coccyx). Spinal stabilization systems can be used to correct problems in the cervical, thoracic, and lumbar portions of the spine, and are often installed posterior to the spine on opposite sides of the spinous process and adjacent to the superior-inferior process. Some implants can be implanted anterior to the spine and some implants can be implanted at other locations as selected by surgical personnel such as at posterior locations on the vertebra.

Often, spinal stabilization may include rigid support for the affected regions of the spine. Such systems can limit movement in the affected regions in virtually all directions. Such spinal stabilizations are often referred to as “static” stabilization systems and can be used in conjunction with techniques intended to promote fusion of adjacent vertebrae in which the boney tissue of the vertebrae grow together, merge, and assist with immobilizing one or more intervertebral joints. More recently, so called “dynamic” spinal stabilization systems have been introduced wherein the implants allow at least some movement (e.g., flexion or extension) of the affected regions of the spine in at least some directions. Dynamic stabilization systems therefore allow the patient greater freedom of motion at the treated intervertebral joint(s) and, in some cases, improved quality of life over that offered by static stabilization systems.

### SUMMARY

Embodiments disclosed herein provide collars for reducing spinal stabilization rods in conjunction with a bone fastener which can attach the collar to various vertebra or other boney structures. Various attachment mechanisms such as threaded members can also be used to attach the collar to various boney structures such as vertebra. The collar can include a body, extension, and a separation member coupling the body and the extension. The collar body and collar extension can each include a pair of arms which correspond to each other. The pairs of arms can define threaded slots which correspond to each other and which can accept spinal stabilization rods and closure members. The separation member can be shaped, dimensioned, or otherwise structured to transmit a tensile force between the collar body and the collar extension which is caused by reducing the spinal stabilization rod into position in the collar. In some embodiments, the tensile force may be at most about 1000 pounds. With regard to torque, the separation member can be configured to yield at a selected torque so that the collar extension can be separated from the collar body by twisting the collar extension relative to the collar body. In some embodiments, the selected yield torque can be no more than about 8.3 foot-pounds.

In some embodiments, the separation member can be a webbing with a first peripheral length substantially shorter than a second peripheral length of the collar body. The separation member can be adjacent to the threaded slots and can define a thread gap which separates the threads of the slots. The collar body can include tensile force relief features near the separation member. Collar bodies and collar extensions of various embodiments can include corners proximal to the separation member. Collar bodies and collar extensions can include anti-splay features associated with their respective threads to mitigate reaction moments which are associated with reducing spinal stabilization rods within the respective collar extensions and which might otherwise be transmitted to the separation member.

In one embodiment, a collar for reducing a spinal stabilization rod is provided. The collar can be a portion of a bone anchor. In some embodiments, the collar can be used in conjunction with a bone fastener for attaching the collar to the vertebra. The collar can include a body, an extension of the body, and a separation member coupling the collar body and the collar extension to each other. Both the collar body and the collar extension can include pairs of arms defining peripheral lengths and threaded slots to accept various spinal stabilization rods and closure members. The separation member can be adjacent to the threaded slots and can define a peripheral length substantially shorter than that of the arms of the collar body and the collar extension. In some embodiments, the separation member can define a thread gap between the threads of the collar body and the collar extension. The separation member can be configured to transmit a tensile force between the collar body and the collar extension. In some embodiments, the tensile force is no more than about 1000 pounds. In some embodiments, the separation member yields at a first selected torque of no more than about 8.3 foot-pounds. Anti-splay features can be included in the threads of the collar extension, the collar body, or both to mitigate reaction moments which can develop during reduction of spinal stabilization rods through the collar extension.

In one embodiment, a method can include various steps for reducing spinal stabilization rods. One such step can be attaching a collar to a vertebra. The collar can include a body and an extension, each of which includes a pair of arms defining a threaded slot. A separation member of the collar can couple the collar body and the collar extension to each other. The collar can be attached to a vertebra by way of a bone fastener. Another step of the method can include reducing the spinal stabilization rod using the collar. The method can also include separating the collar extension from the collar body by applying a torque to the collar extension to cause the separating member to yield and, ultimately to, fail in torsion.

Embodiments disclosed herein provide many advantages. For example, the separation of the collar extension from the collar body can be accomplished with one substantially continuous motion rather than repeated application of bending moments to the arms of the collar extension. Thus, the separation of the collar extension from the collar body can leave patient tissues which may be adjacent to the collar substantially undisturbed. In some embodiments, the arms of the collar extension can be prevented from splaying, thereby mitigating reaction moments which might otherwise be transmitted to the separation member during the reduction of the spinal stabilization rod through portions of the collar extension.

### BRIEF DESCRIPTION OF THE DRAWINGS

A more complete understanding of the present disclosure and additional advantages thereof may be acquired by referring to the following description, taken in conjunction with the accompanying drawings in which like reference numbers indicate like features and wherein:

FIG. 1 depicts a human axial skeleton including a skull and spine.

FIG. 2A depicts one embodiment of a spinal stabilization system for treating various conditions affecting the spine.

FIG. 2B depicts one embodiment of a spinal stabilization system for treating various conditions affecting the spine.

FIG. 2C depicts one embodiment of a closure member for educing a spinal stabilization rod.

FIG. 2D depicts one embodiment of a spinal stabilization system for treating various conditions affecting the spine.

FIG. 3 depicts one embodiment of a bone anchor for reducing a spinal stabilization rod.

FIG. 4 depicts one embodiment of a collar for reducing a spinal stabilization rod.

FIG. 5 depicts one embodiment of a collar for reducing a spinal stabilization rod.

FIG. 6 depicts one embodiment of a collar for reducing a spinal stabilization rod.

### DETAILED DESCRIPTION

The disclosure and the various features and advantageous details thereof are explained more fully with reference to the non-limiting embodiments detailed in the following description. Descriptions of well known starting materials, manufacturing techniques, components, and equipment are omitted so as not to unnecessarily obscure the disclosure in detail. Skilled artisans should understand, however, that the detailed description and the specific examples, while disclosing preferred embodiments of the disclosure, are given by way of illustration only and not by way of limitation. Various substitutions, modifications, and additions within the scope of the underlying inventive concept(s) will become apparent to those skilled in the art after reading this disclosure. Skilled artisans can also appreciate that the drawings disclosed herein are not necessarily drawn to scale.

As used herein, the terms “comprises,” “comprising,” “includes,” “including,” “has,” “having” or any other variation thereof, are intended to cover a non-exclusive inclusion. For example, a process, process, article, or apparatus that comprises a list of elements is not necessarily limited only those elements but may include other elements not expressly listed or inherent to such process, process, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Additionally, any examples or illustrations given herein are not to be regarded in any way as restrictions on, limits to, or express definitions of, any term or terms with which they are utilized. Instead, these examples or illustrations are to be regarded as being described with respect to one particular embodiment and as illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example”, “for instance”, “e.g.”, “in one embodiment”.

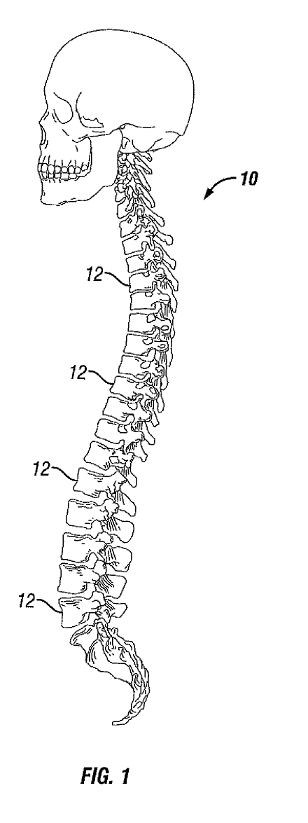

FIG. 1 depicts a human axial skeleton including a skull (composed of numerous cranial bones (such as parietal bones, temporal bones, zygomatic bones, mastoid bones, maxilla bones, mandible bones, etc.) and spine 10 including numerous vertebrae 12 and intervertebral disks separating certain vertebrae 12. Ligaments can join vertebrae 12 and muscles and tendons can attach to vertebrae 12. As discussed previously, spine 10 carries loads imposed on the patient's body and loads generated by the patient. Vertebrae 12 cooperate to allow spine 10 to extend, flex, rotate, etc. under the influence of various muscles, tendons, ligaments, etc. attached to spine 10. Spine 10 can also cooperate with various muscles, tendons, ligaments, etc. to cause other anatomical features of the patient's body to move and exert force on objects in the environment.

However, certain conditions can cause damage to spine 10, vertebrae 12, certain intervertebral discs, etc. and can impede the ability of spine 10 to move in various manners. These conditions include, but are not limited to abnormal curvature, injury, infections, tumor formation, arthritic disorders, puncture, or slippage of the intervertebral disks, and injuries or illness such as spinal stenosis and prolapsed disks. As some of these conditions progress, come into existence, or persist, various symptoms can indicate the desirability of stabilizing spine 10 or curing deformities thereof. As a result of these various conditions, the ability of the patient to move, with or without pain or discomfort, can be impeded. Additionally, certain physiological processes such as breathing can be impeded by such conditions. Based on various indications, medical personnel might recommend attaching one or more spinal stabilization or correction systems (hereinafter spinal stabilization systems) to vertebra 12 of spine 10 (among other possible remedial actions such as physical therapy) to correct the particular condition(s) from which the patient may be suffering and to prevent further deterioration of spine 10, vertebra 12, etc.

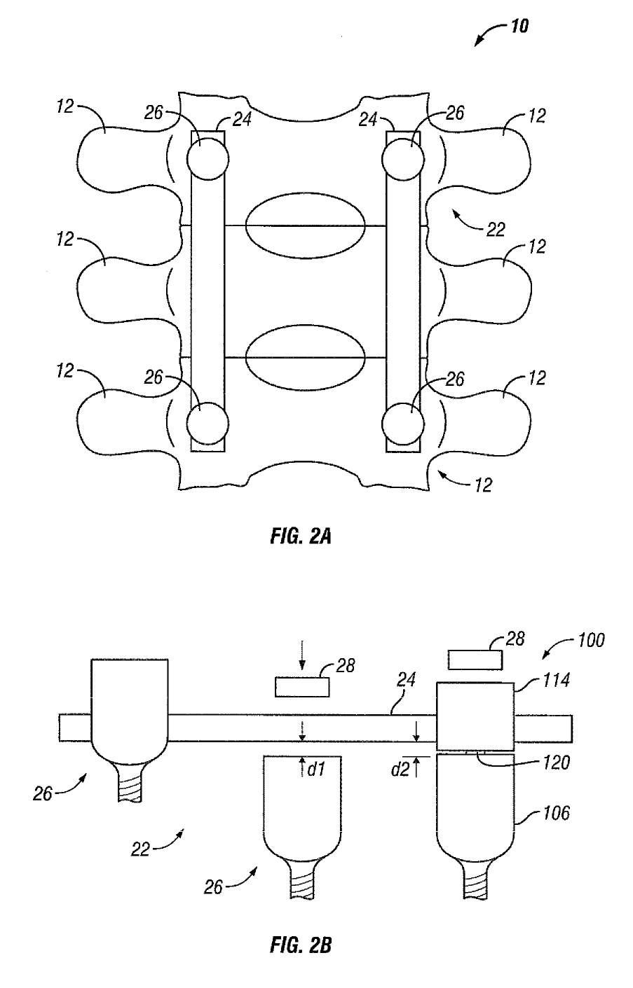

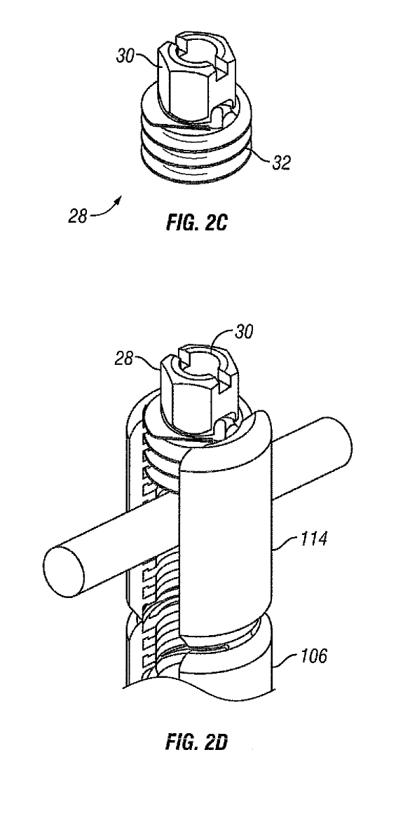

FIG. 2A depicts a side elevation view of a portion of spine 10 including various vertebrae 12, inter-vertebral disks, spinous processes, vertebral facets, intravertebral area, etc. For clarity, FIG. 2A omits the muscles, tendons, ligaments, etc. that might be associated with spine 10. FIG. 2A also depicts spinal stabilization system 22 which can include a pair of spinal stabilization rods 24 (only one is shown in FIG. 2A) and bone fastener assemblies 26 which can attach spinal stabilization rods 24 to vertebra 12 or other boney structures. One particular spinal stabilization rod 24 and its associated bone fastener assemblies 26 can be attached to vertebrae 12 on one side of a particular spinous process 16 while the other spinal stabilization rod 24 can be attached to vertebrae 12 on the other side of spinous process 16. Spinal stabilization rods 24 can be static (allowing little or no relative motion between adjacent vertebrae 12 of spine 10) or dynamic (allowing at least some relative motion between adjacent vertebrae 12 of spine 10). As will be discussed with more particularity herein, bone anchors of various embodiments can be used to attach spinal stabilization system 22 to vertebrae 12 as well as other boney structures.

It may be helpful at this juncture to briefly describe various aspects of vertebrae 12 (see FIG. 1). For instance, potential attachment points for spinal stabilization system 22 can include superior-inferior processes, transverse processes, vertebral facets, various surfaces exposed by surgical personnel removing portions of vertebrae 12, etc. Spinous processes and superior-inferior processes allow tendons, muscles, etc. to attach to spine 10 for movement of spine 10 and various anatomical structures which are attached to spine 10 or which may be affected thereby. These anatomical structures can include the patient's ribs, hips, shoulders, head, legs, etc. Spinous processes extend generally in a posterior and slightly inferior direction from vertebrae 12. Superior-inferior processes are also boney structures and extend generally laterally from vertebrae 12 and allow ligaments, muscles, and tendons to attach to vertebra 12. Vertebral facets join adjacent vertebrae 12 to each other while allowing motion there between by being in sliding contact with corresponding vertebral facets of these adjacent vertebrae 12.

With continuing reference to FIG. 2A, during certain types of motion of spine 10 (such as flexing and extending) which can be caused (or resisted) by various muscles, vertebrae 12 tend to rotate relative to each other about axes of rotation generally in intravertebral areas. Intravertebral areas can be adjacent to and posterior to intervertebral disks and substantially anterior to spinous processes and vertebral facets. Since vertebral facets allow vertebrae to articulate about these axes of rotation little or no reactionary forces or moments are generated by healthy spines 10 during ordinary movements.

However, due to various chronic, degenerative, genetic, etc. conditions, spine 10 can be deformed and, in some cases, deformed in significant manners. With reference to FIG. 2A, in some instances, spine 10 can be deformed sufficiently that spinal stabilization rod 24, when seated in one particular bone fastener assembly 26, happens to be spaced apart from certain other bone fastener assemblies 26 by some distance. In such cases, spinal stabilization rod 24 can be said to be “proud” of these other bone fastener assemblies 26 by such distances. When spinal stabilization rod 24 is proud of particular bone fastener assemblies 26 but still remains within at least one other bone fastener assembly 26, internal threads of this other bone fastener assembly 26 can allow closure member 28 to be driven down and into that other bone fastener assembly 26, thereby reducing spinal stabilization rod 24 into its intended position in that bone fastener assembly 26.

In some situations, it may be difficult for surgical personnel to reduce spinal stabilization rod 24 into the particular bone fastener assembly 26. For instance, if spinal stabilization rod 24 happens to be proud of its intended position in a particular bone fastener assembly 26 by a distance which places it outside of the particular bone fastener assembly 26, closure member 28 cannot engage the internal threads of the particular bone fastener assembly 26 to reduce spinal stabilization rod 24 into position. Moreover, because spine 10 and the anatomical features coupled thereto may resist movement of spinal stabilization rod 24, it may be difficult for surgical personnel to force spinal stabilization rod 24 into the particular bone fastener assembly 26 absent certain mechanical advantages provided by internal threads of bone fastener assembly 26. Previously, surgical personnel might have had to remove spinal stabilization rods 24, bone fastener assemblies 26, and potentially other portions of spinal stabilization system 22 and attempt to stabilize spine 10 with different spinal stabilization system components or different spinal stabilization systems altogether. Because of the additional surgical steps (and surgical operating time associated there with), the patient can suffer additional trauma to tissues in (or adjacent to) the surgical site, post-operative complications, discomfort, pain, etc.

FIG. 2B depicts a side view of one embodiment of spinal stabilization system 22. In FIG. 2B, spinal stabilization system 22 includes bone screws 26, bone anchor 100, and spinal stabilization rod 24. Bone screws 26 and bone anchor 100 can be monoaxial (adapted to hold spinal stabilization rods 24 at some particular orientation) or polyaxial (adapted to hold spinal stabilization rods 24 at various orientations). Bone screws 26 and bone anchor 100 may be assembled from other separate components or can be formed as continuous components themselves. For clarity, boney structures to which bone screws 26 and bone anchor 100 may be attached are not shown.

Bone anchor 100 can include anchor body 106, anchor extension 114, and separation member 120 coupling anchor body 106 and anchor extension 114 to each other. Thus, bone anchor 100 includes anchor extension 114 of anchor body 106 whereas bone screws 26 include screw bodies but do not include screw extensions 114. Accordingly, bone anchor 100 can reduce spinal stabilization rods 24 which would be proud of bone screw 26 should bone screw 26 be used in the particular position in spinal stabilization system 22 at which bone anchor 100 happens to be located. More particularly, spinal stabilization rod 24 is shown in FIG. 2B as being proud of a particular bone screw 26 and bone anchor 100 by distances d1 and d2 respectively. Whereas bone anchor 100 can reduce spinal stabilization rod 24 through distance d2 (because spinal stabilization rod 24 lies within a threaded slot of anchor extension 114), bone screw 26 cannot reduce spinal stabilization rod 24 through distance d1 (because spinal stabilization rod 24 lies outside of bone screw 26).

FIG. 2B further illustrates that bone anchors 100 can be used to reduce spinal stabilization rods 24 in some situations in which bone screws 26 can not be used to reduce spinal stabilization rods 24. In spinal stabilization system 22, spinal stabilization rod 24 has been reduced into the particular bone screw 26 at one end of spinal stabilization system 22. Spinal stabilization rod 24 is shown as being proud of the other bone screw 26′ by distance d1. It can be very difficult, if not impossible, to reduce spinal stabilization rod 24 into bone screw 26. Spinal stabilization rod 24 is also shown as being proud of anchor body 106 of bone anchor 100 by distance d2. Spinal stabilization rod 24, however, is in anchor extension 114 of bone anchor 100. Thus, spinal stabilization rod 24 can be reduced distance d2 into anchor body 106 by surgical personnel using closure member 28 in conjunction with anchor extension 114. It may therefore be desirable to use bone anchors 100 with anchor extensions 114 to reduce spinal stabilization rods 24 through greater distances 21 and d2 than possible with bone screws 26 in more than one location in spinal stabilization system 22.

FIG. 2C depicts a perspective view of closure member 28 for a bone fastener assembly 26. Closure member 28 may include tool portion 30 and male threading 32. Tool portion 30 may couple to a tool that allows closure member 106 to be positioned in a collar of bone fastener assembly 26 or 100. Tool portion 30 may include various configurations (e.g., threads, hexalobular connections, hexes) for engaging a tool (e.g., a driver). Male threading 32 may have a shape that complements the shape of female threading in f anchor body 106 and anchor extension 114.

FIG. 2D depicts a perspective view of a portion of a spinal stabilization system having closure member 28 of FIG. 2C resting on top of spinal stabilization rod 24 which is seated in extension 114 of a bone anchor 100. Closure member 28 may couple to extension 114 by a variety of systems including, but not limited to, standard threads, modified threads, reverse angle threads, buttress threads, or helical flanges. Closure member 28 may be advanced into an opening in bone anchor 100 to engage a portion of spinal stabilization rod 24. In some embodiments, closure member 28 may inhibit movement of spinal stabilization rod 24 relative to bone anchor 100.

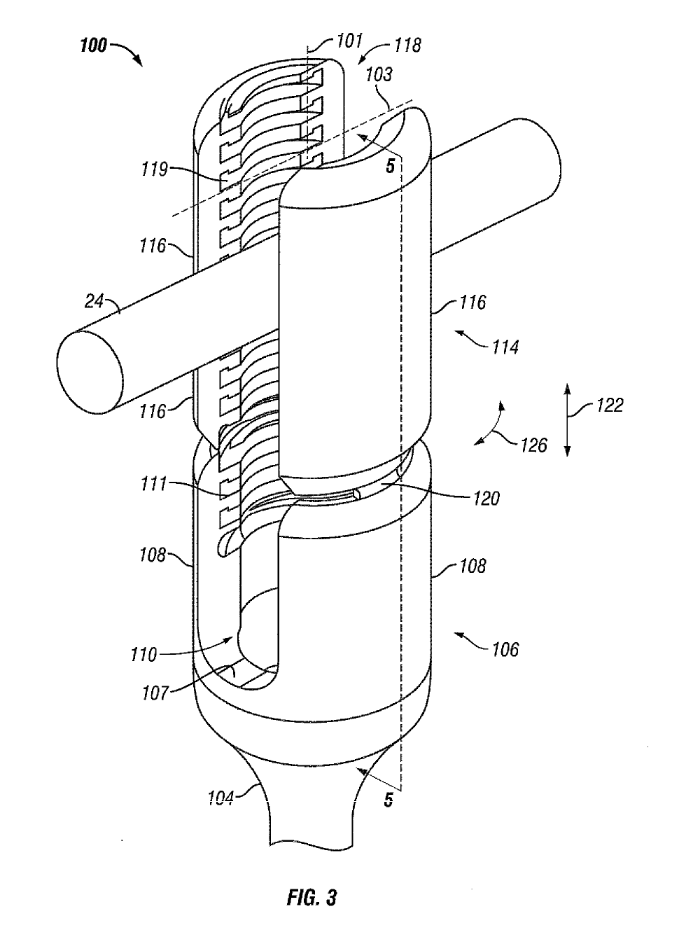

With reference now to FIG. 3, FIG. 3 depicts a perspective view of one embodiment of bone anchor 100. Bone anchor 100 can be used in conjunction with closure member 28 (see FIG. 2B) to reduce spinal stabilization rod 24. As shown in FIG. 3, bone anchor 100 can include threaded member 104, anchor body 106, and anchor extension 114. Threaded member 104 can be coupled to the distal end of anchor body 106. Threaded member 104 can be used to attach bone anchor 100 to various boney structures such as vertebrae 12 during spinal stabilization operations. Anchor body 106 can define spinal stabilization rod seat 107 for spinal stabilization rod 24 and can include a pair of arms 108. Arms 108 of anchor body 106 can define threaded slot 110 of anchor body 106 and can form the walls of threaded slot 110 of anchor body 106. The walls of threaded slot 110 of anchor body 106 can include threads 111 of anchor body 106. Bone anchor 100 can include anchor extension 114 with a pair of arms 116 which define threaded slot 118 of anchor extension 114. Arms 116 of anchor extension 114 can form the walls of threaded slot 118 of anchor extension 114. The walls of threaded slot 118 of anchor extension 114 can define threads 119 of anchor extension 114.

With further reference to FIG. 3, FIG. 3 shows that bone anchor 100 may have longitudinal axis 101 extending in a direction between anchor body 106 and anchor extension 114. Bone anchor 100 can include any type of attachment member (not shown) to attach bone anchor 100 to various boney structures. Bone anchor 100 can also have superior-inferior axis 103 extending in a direction perpendicular to longitudinal axis 101. Herein, the term “torque” can refer to forces which tend to cause rotation about axes generally parallel with longitudinal axis 101. The term “moment”, herein, can refer to forces which tend to cause rotation about axes generally parallel to superior-inferior axis 103 (or any axis perpendicular to longitudinal axis 101 such as a medial-lateral axis). Separation member 120 can couple anchor body 106 and anchor extension 114 to each other, thereby transmitting tensile forces 122 (and compressive forces) generally parallel to longitudinal axis 101, torques, and moments (up to selected force, torque, and moment levels in some embodiments) between anchor body 106 and anchor extension 114. Together, various anchor bodies 106, anchor extensions 114, and separation members 120 can form collars for reducing spinal stabilization rods 24 used in spinal stabilization systems 22.

As noted previously, threaded member 104 can attach bone anchor 100 to vertebrae 12 (see FIG. 1). Anchor body 106 can be adjacent to threaded member 104 such that anchor body 106 can be used to reduce spinal stabilization rods 24 which lie proximally to vertebrae 12. Separation member 120 can be interposed between anchor body 106 and anchor extension 114 to couple anchor body 106 and anchor extension 114 and to allow separation of anchor extension 114 from anchor body 106. In some embodiments, separation member 120 can be spaced apart from threaded slot 110 of anchor body 106 and threaded slot 118 of anchor extension 114 and can be oriented substantially parallel to superior-inferior axis 103. Separation member 120, however, can be positioned at any location along the proximal end of anchor body 106 and anchor extension 114 as desired.

Anchor extension 114 can be adjacent to separation member 120 and on the opposite side of separation member 120 from anchor body 106. Thus, anchor extension 114 can be used to reduce spinal stabilization rods 24 from proud positions beyond anchor body 106 to proud positions within anchor body 106. Threads 111 of anchor body 106 can be continuous along the length of anchor body 106 and threads 119 of anchor extension 114 can be continuous along the length of anchor extension 114. Together, threads 111 of anchor body 106 and threads 119 of anchor extension 114 can provide a substantially continuous threaded path from the proximal end of anchor extension 114 to the distal end or anchor body 106.

Thus, threaded slot 118 of anchor extension 114 can accept spinal stabilization rods 24 and allow closure members 28 (in conjunction with threads 119 of anchor extension 114) to reduce spinal stabilization rods 24 through anchor extension 114, passed separation members 120, and into anchor body 106. At some time, closure member 28 translates sufficiently far through threaded slot 118 of anchor extension 114 that closure member 28 disengages from threads 119 of anchor extension 114 (at the distal end of threaded slot 118 of anchor extension 114). Closure member 28 can, at about that time, engage threads 111 of anchor body 106. Closure member 28 can therefore continue reducing spinal stabilization rod 24 toward the distal end of threaded slot 110 of anchor body 106. As a result, closure member 28 can reduce spinal stabilization rods from proud positions in threaded slot 118 of anchor extension 114 and seat spinal stabilization rods 24 against spinal stabilization rod seat 107 of bone anchor 100.

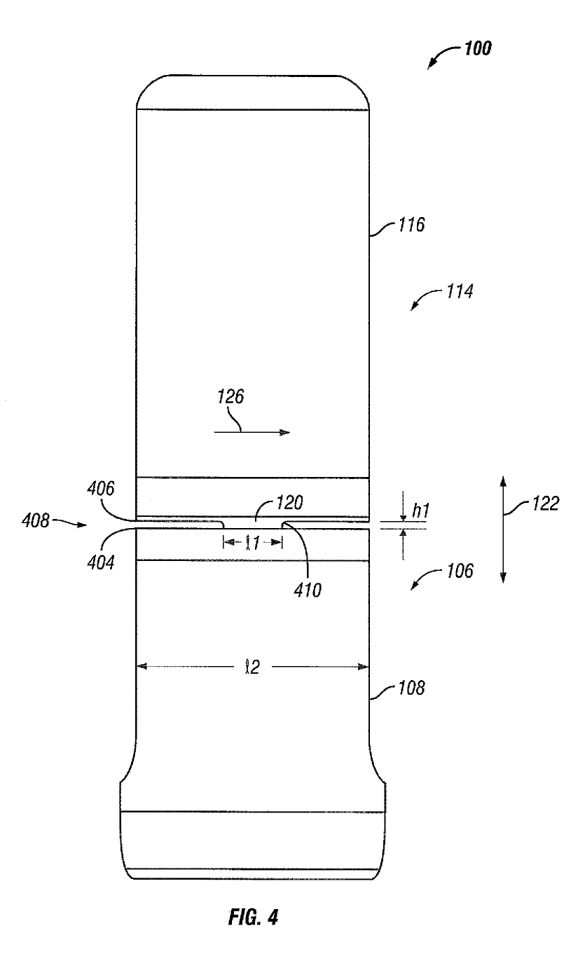

With reference now to FIG. 4, FIG. 4 depicts a side elevation view of one embodiment of bone anchor 100. FIG. 4 illustrates anchor body 106, arm 108 of anchor body 106, anchor extension 114, arm 116 of anchor extension 114, and separation member 120 coupling anchor body 106 and anchor extension 114 to each other. FIG. 4 also illustrates that anchor body 106 can include corner 404 on its proximal end and generally adjacent to anchor extension 114 and separation member 120. Anchor extension 114 can include corner 406 on its distal end and generally adjacent to anchor body 106 and separation member 120.

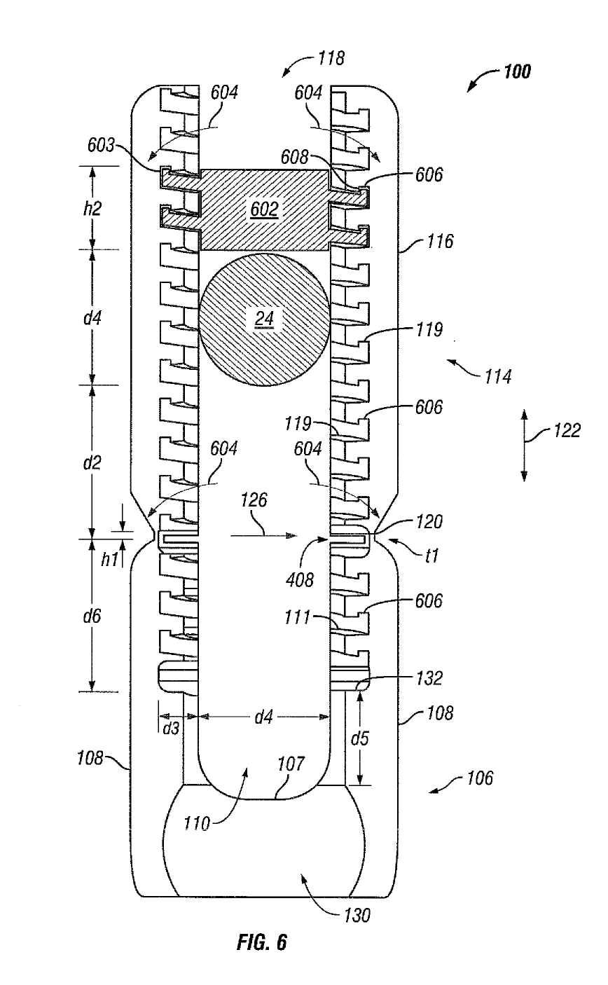

Separation member 120 can be of height h1 and peripheral length l1. Peripheral length l1 of separation member 120 can be substantially less than peripheral length l2 of arm 108 of anchor body 106. Thus, separation member 120 can define thread gap 408 between anchor body 106 and anchor extension 114 of height h1 and length l2-l1 (or some fraction thereof such as ½). Thread gap 408 is discussed further with reference to FIGS. 6 and 7.

Tensile stress relief features 410 (such as filets) can be included adjacent to and adjoining separation member 120 to relieve tensile stresses that might otherwise concentrate adjacent to, or within, separation member 120. Features other than the particular filet shown as tensile force relief feature 140 can be employed to relieve tensile forces 122 and compressive forces in the proximity of separation member 120. Stress risers may also be included in bone anchor 100 generally adjacent to and adjoining separation member 120 to concentrate select types of stress (such as torsional stresses) within, or in, the proximity of separation member 120.

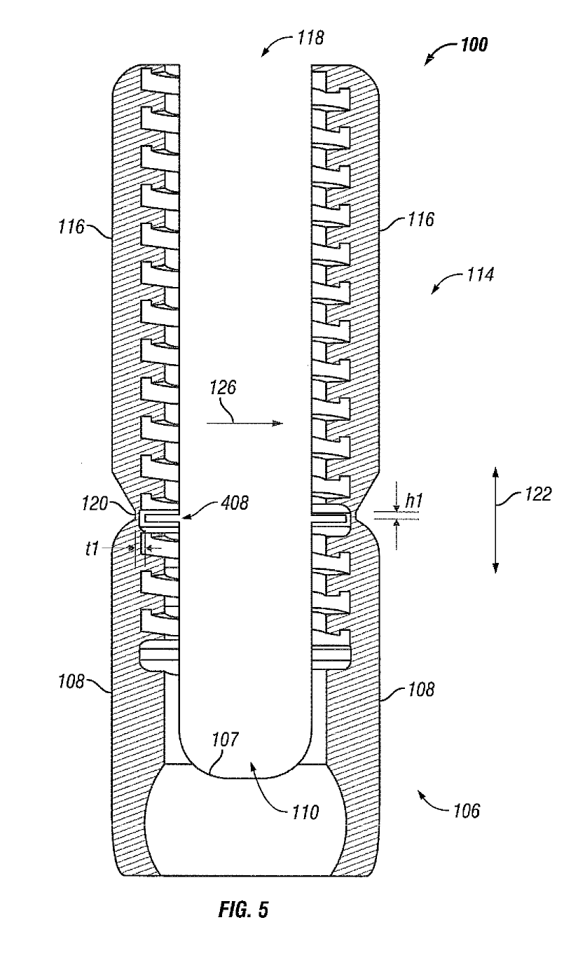

With reference now to FIG. 5, FIG. 5 depicts a cross sectional view taken along line 5-5 of FIG. 4 of one embodiment of bone anchor 100. FIG. 5 illustrates anchor body 106, spinal stabilization rod seat 107, arms 108 of anchor body 106, anchor extension 114, arms 116 of anchor extension 114, and separation members 120 coupling anchor body 106 and anchor extension 114 to each other. Separation member 120 can be of thickness t1 as illustrated in FIG. 5. Thus, separation member 120 (being of peripheral length l1 height h1, and thickness t1) can define thread gap 408 in conjunction with peripheral lengths 12 of arms 108 of anchor body 106 and arms 116 of anchor extension 114 (the lengths of which can differ in some embodiments).

Peripheral length l1, height h1, thickness t1, various stress relief (and riser) features 410 (see FIG. 4), and material properties of anchor body 106, anchor extension 114, and separation member 120 can define the tensile and compressive forces, torques, and moments (the latter being discussed further with reference to FIG. 6) which separation member 120 can transmit between anchor body 106 and anchor extension 114 without failing or yielding. Peripheral length l1, height h1, thickness t1, various stress relief (and riser) features 410 (see FIG. 4), and material properties of anchor body 106, anchor extension 114, and separation member 120 can define the tensile and compressive forces, torque, and moments which can cause separation member 120 to yield, and ultimately to fail, when such forces are applied to anchor body 106 and anchor extension 114 and transmitted to separation member. Thus, separation member 120 can be adapted to fail under selected tensile or compressive forces, torque, or moments as desired. By failing in one or more of theses modes (such as torsional), separation member can allow anchor extension 114 to be separated from anchor body 106.

FIG. 6 depicts a cross sectional view of one embodiment of bone anchor 100 with spinal stabilization rod 24 and closure member 602 positioned in threaded slot 118 of anchor extension 114. FIG. 6 also shows anchor body 106, arms 108 of anchor body 106, threaded slot 110 of anchor body 106, threads 111 of anchor body 106, anchor extension 114, arms 116 of anchor extension 114, threaded slot 118 of anchor extension 114, threads 119 of anchor extension 114, and separation member 120 (having height h1 and thickness t1). Spinal stabilization rod 24 is illustrated as being proud of anchor body 106 by distance d2 although spinal stabilization rod could be at any position along the length of threaded slots 110 and 118. As a result of the proud position of spinal stabilization rod 24 relative to threaded slot 110 of anchor body 106, and in the absence of anchor extension 114, surgical personnel would have difficulty in reducing bone anchor 100 into anchor body 106.

With bone anchor 100 including anchor extension 114 though, surgical personnel can reduce spinal stabilization rod 24 into anchor body 106 through distance d2. More particularly, surgical personnel can drive closure member 602 through threaded slot 118 of anchor extension 114 using an appropriate tool such as a screwdriver. As surgical personnel drive closure member 602 through slot 118 of anchor extension 114, threads 603 of closure member 602 (which can correspond to threads 111 of anchor body 106 and threads 119 of anchor extension 114) can engage threads 119 of anchor extension 114. Spinal stabilization rod 24 often resists the urging of closure member 602 toward anchor body 106 as closure member 602 is being driven through bone anchor 100 because of forces generated by spinal stabilization system 22 (see FIG. 2) and anatomical features to which spinal stabilization 22 may be attached. Thus, driving closure member 602 toward anchor body 106 can place bone anchor 100 in tension as indicated by arrow 122. Tensile force 122 can be transmitted between anchor body 106 and anchor extension 114 by separation member 120. Separation member 120 can be sized, shaped, etc. to withstand such tensile forces 122.

In addition to placing bone anchor 100 in tension, the act of driving closure member 602 against forces exerted on it by spinal stabilization rod 24 can cause a reaction at arms 116 of anchor extension 114 which can include reaction moments 604. Reaction moments 604 can cause arms 116 of anchor extension 114 to splay (move apart from each other). Since separation member 120 can be coupled to arms 116 of anchor extension 114, reaction moments 604 can be transmitted to separation member 120. Separation member 120 can be sized, shaped, etc. to resist such reaction moments 604.

In some embodiments, anchor extension 114 includes anti-splay contour 606 while closure member 602 includes anti-splay contour 608. Anti-splay contour 606 of anchor extension 114 and anti-splay contour 608 of closure member 602 can mechanically interlock and resist reaction moments 604 and the tendency to splay arms 116 of anchor extension 114 caused thereby. Because anti-splay contours 606 of anchor extension 114 and anti-splay contour 608 of closure member 602 can resist reaction moments 604, anti-splay contour 606 of anchor extension 114 and anchor 608 of closure member 603 can reduce, if not eliminate, reaction moments 604. Thus, anti-splay contour 606 of anchor extension 114 and anti-splay contour 608 of closure member 602 can reduce reaction moments 604 which might otherwise be transmitted to separation member 120. Accordingly, selection of various properties and characteristics associated with the abilities of separation member 120 to resist tensile and compressive forces, torques, etc. (and to fail when such selected forces, or greater, are applied to it) can be simplified as a result of including anti-splay contour 606 of anchor extension 114 and anti-splay contour 608 of closure member 602 in spinal stabilizations systems (such as spinal stabilization system 22 of FIG. 2B).

With continuing reference to FIG. 6, as surgical personnel drive closure member 602 toward anchor body 106, threads 603 of closure member 602 can encounter thread gap 408. Thread gap 408, threads 111 of anchor body 106, and threads 119 of anchor extension 114 can be configured so that threads 111 of anchor body 106 and threads 119 of anchor extension 114 form a substantially continuous thread along threaded slot 110 of anchor body 106 and threaded slot 118 of anchor extension 114. Threads 603 of closure member 602 can have a length exceeding height h1 in some embodiments. Nevertheless, height h1 of thread gap 408 can be minimized in some embodiments. Thus, despite thread gap 408, threads 603 of closure member 602 can remain substantially fully engaged with threads 119 of anchor extension 114, threads 111 of anchor body 106, or a combination thereof.

As threads 603 of closure member 602 engage threads 111 of anchor body 106, reaction moments 604 can be spread between threads 119 of anchor extension 114 and threads 111 of anchor body 106. Reaction moments acting on threads 119 of anchor extension 114 can therefore begin to decrease in anchor extension 114. Correspondingly, reaction moments 604 acting in threads 111 of anchor body 106 and in anchor body 106 can begin to increase as closure member 602 is driven across thread gap 408. As a result, reaction moments 604 transmitted to separation member 120 can decrease until closure member 602 is driven across thread gap 408 and threads 603 of closure member 602 disengage from threads 119 of anchor extension 114. Reaction moments 604 in anchor extension 114 and separation member 120 can become essentially zero as threads 603 of closure member 602 completely disengage from threads 119 of anchor extension 114.

As surgical personnel drive closure member 602 through anchor body 106, spinal stabilization rod 24 can translate toward the distal end of bone anchor 100. At some time, spinal stabilization rod 24 can seat in spinal stabilization rod seat 107 of the distal end of anchor body 106. The interaction between threads 111 of anchor body 106 and threads 603 of closure member 602 can hold closure member 602 in place.

Surgical personnel can apply some selected torque to anchor extension 114 as indicated by arrow 126. With anchor body 106 attached to some boney structure such as vertebra 12 (see FIG. 1), or otherwise held static relative to anchor extension 114, selected torque 126 can cause separation member 120 to fail in torsion. As separation member 120 fails, separation member 120 can allow anchor extension 114 to separate from anchor body 106. Thus, bone anchor 100, with spinal stabilization rod 24 seated in it, can provide spinal stabilization system 22 (see FIG. 1) with a low profile when implanted in a patient.

In some embodiments, separation members 120 can therefore be adapted (by selection of appropriate dimensions, material properties, etc.) to transmit tensile forces 122 (and compressive forces) between anchor body 106 and anchor extension 114. Separation member 120 can be adapted to yield at selected torque 126 yet still transmit torque below selected torque 126 between anchor body 106 and anchor extension 114. Separation members 120 can be adapted to transmit reaction moments 604 between anchor body 106 and anchor extension 114 although separation members 120 need not be adapted to transmit reaction moments 604 between anchor body 106 and anchor extension 114. Anti-splay contour 606 of anchor extension 114 and anti-splay contour 608 of closure member 602 can be configured to mitigate reaction moments 604 transmitted to separation member 120 as desired.

In some embodiments, separation member 120 can allow bone anchor 100 to be attached to vertebrae 12 and other boney structures by the action of surgical personnel placing threaded member 104 (see FIG. 3) on such structures and advancing bone anchor 100 along longitudinal axis 101. While surgical personnel advance bone anchor 100, surgical personnel can apply torque up to selected torque 126 to anchor extension 114 to tap threads of threaded member 104 into the boney structure. Surgical personnel may, if desired, temporarily thread closure member 602 into threaded slot 118 of anchor extension 114 before applying torque to anchor extension 114 to provide greater structural strength to anchor extension 114 during attachment of bone anchor 100 to vertebrae 12.

In some embodiments, bone anchor 100 can be attached to boney structures by applying torque to anchor body 106 instead of anchor extension 114. Instruments can be used to aid surgical personnel in applying such torque to anchor body 106 if desired. Thus, separation member 120 need not be configured to transmit torques beyond some selected separation torque between anchor extension 114 and anchor body 106.

With continuing reference to FIG. 6, FIG. 6 illustrates that threads 111 of anchor body 106 can have depth d3. Threads 119 of anchor extension 114 can correspond to threads 111 of anchor body 106 and, therefore, can also have depth d3. Depth d3 can allow threads 603 of closure member 602 to fully engage threads 111 of anchor body 106 and threads 119 of anchor extension 114. Threads 111 of anchor body 106 and threads 119 of anchor extension 114 can be formed in bone anchor 100 in a continuous fashion so that threads of any particular member that engages threads 111 of anchor body 106 or threads 119 of anchor extension 114 can engage the other threads. As noted previously, it may be desired to drive closure member 602 across thread gap 408. Thus, separation members 120 can be positioned some distance equal to or greater than thread depth d3 away from threaded slot 110 of anchor body 106 and threaded slot 118 of anchor extension 114. Thus, separation members 120 can be positioned to avoid interference with threads 603 of closure member 602 as closure member 602 is driven across thread gap 408. In some embodiments, separation member 120 can extend into thread gap 408 and can define threads corresponding to, and aligned with, threads 111 of anchor body 106 and threads 119 of anchor extension 114.

FIG. 6 also depicts threaded member socket 130. Threaded member socket 130 can be used to assemble threaded member 104 (see FIG.) to bone anchor 100. More particularly, threaded member 104 can include a head portion (not shown) corresponding in shape to threaded member socket 130. Threaded member 104 and threaded member socket 130 can include features to lock threaded member 104 to bone anchor 100 while providing certain degrees of freedom of movement between threaded member 104 and bone anchor 100. Such features can also limit the number of degrees of freedom threaded member 104 possesses to move relative to bone anchor 100. Thus, bone anchor 100 can be a monoaxial screw, a polyaxial screw, etc. without departing from the scope of the disclosure. U.S. patent application Ser. No. 12/186,446, entitled SPINAL STABILIZATION SYSTEMS AND METHODS, by Crall et el., and filed on Aug. 5, 2008, discloses various polyaxial and monoaxial bone anchors and is incorporated herein as if set forth in full.

Still with reference to FIG. 6, FIG. 6 also depicts spinal stabilization rod 24 as having diameter d4. Diameter d4 can be equal to a distance d5 between spinal stabilization rod seat 107 and the distal end of threads 111 of anchor body 106. Furthermore, closure member 602 can have a height h2, with threads 111 of anchor body 106 being a distance d6 in length. In some embodiments, distance d6 of threads 111 of anchor body 106 can be equal to or greater than closure member 602 height h2. Anchor body 106 can define hard stop 132 at the distal end of threads 111 of anchor body 106 so that closure member 602 can be driven a select distance d6 into anchor body 106. Thus, spinal stabilization rod 24 can be seated on spinal stabilization rod seat 107 with closure member 602 approximately fully driven into threaded slot 110 of anchor body 106 and seated in spinal stabilization rod seat 107.

With continuing reference to FIG. 6 bone anchor 100 can be attached to boney structures such as vertebra and used to reduce spinal stabilization rods 24 in various fashions. In one embodiment, the attachment of bone anchor 100 to a particular boney structure can be by way of applying a torque to either anchor body 106 or anchor extension 114 and advancing threaded member 104 into the boney structure. If desired, closure member 602 can be in threaded slot 110 of anchor body 106 or threaded slot 118 of anchor extension 114 during step 704 to provide structural strength to anchor body 106 or anchor extension 114. If so, closure member 602 can be withdrawn from threaded slot 110 of anchor body 106 or threaded slot 118 of anchor extension 114. Spinal stabilization rod 24 can be placed in proximity to anchor body 100. Should spinal stabilization rod 24 be placed in a position which is proud of anchor body 106, threaded slot 118 of anchor extension 114 may be aligned with, and positioned about spinal stabilization rod 24. Should spinal stabilization rod 24 be placed in a position proud of spinal stabilization rod seat 107 of anchor body 106, and yet happen to be within threaded slot 110 of anchor body 106, aligning spinal stabilization rod 24 with threaded slot 118 of anchor extension 114 need not be performed.

When desired, closure member 602 can be placed in threaded slot 118 of anchor extension 114 and driven through threaded slot 118 of anchor extension 114. Threads 603 of closure member 602 can engage threads 119 or anchor extension 114 while closure member 602 is being driven in through threaded slot 118 of anchor extension 114. When closure member 602 encounters spinal stabilization rod 24, closure member 602 can begin reducing spinal stabilization rod 24 toward spinal stabilization rod seat 107 of anchor body 106. As closure member 602 reduces spinal stabilization rod 24 toward spinal stabilization rod seat 107, bone anchor 100 can be placed in tension by the interaction of spinal stabilization rod 24 and closure member 602 at step 712 acting through threads 603 of closure member 602 and threads 119 of anchor extension 114 (or threads 111 of anchor body 106). Tensile force 122 arising there from can be transmitted between anchor body 106 and anchor extension 114 by separation member 120.

During reduction of spinal stabilization rod 24, reaction moments 604 can also develop at arms 116 of anchor extension 114 thereby possibly causing arms 116 of anchor extension 114 to splay. Reaction moments 604 can be transmitted to separation member 120 through arms 116 of anchor extension 114. When bone anchor 100 includes anti-splay contour 606 of anchor extension 114 and when closure member 602 includes anti-splay contour 608, anti-splay contour 606 of anchor extension 114 and anti-splay contour 608 of closure member 602 can interlock thereby mitigating reaction moments 604. Mitigating reaction moments 604 can prevent splaying of arms 116 of anchor extension 114. Mitigating reaction moments 604 can also cause a corresponding reduction of moments applied to separation members 120. Whether reaction moments 604 are mitigated by inclusion of anti-splay contour 606 of anchor extension 114 and inclusion of anti-splay contour 608 of closure member 602 or not, any tools used to drive closure member 602 can be removed from threaded slot 110 of anchor body 106 and threaded slot 118 of anchor extension 114. The frictional interaction of threads 111 of anchor body 106 and threads 603 of closure member 602 can lock closure member 602 in place in anchor body 106 with spinal stabilization rod 24 seated in spinal stabilization rod seat 107.

Torque can be applied to anchor extension 114 (or anchor body 106). When the applied torque reaches selected torque 126, separation member 120 can fail thereby allowing anchor extension 114 to separate from anchor body 106 at step 720. The separation of anchor extension 114 from anchor body 106 can be by way of one continuous motion instead of repeatedly bending arms 116 of anchor extension 114 back-and-forth to cause them to fail in (moment induced bending) fatigue. Thus, surgical time spent separating arms 116 of anchor extension 114 from anchor body 106 can be reduced. Furthermore, because separation of anchor extension 114 from anchor body 106 can cause anchor extension 114 to rotate relative to anchor body 106 substantially without translating in medial-lateral, superior-inferior, or posterior-anterior directions, tissues and other anatomical features which might be proximal to bone anchor 100 can be left undisturbed.

In some embodiments, though, some moment can be applied to arms 116 of anchor extension 114 first in one direction and can then be repeatedly reversed until separation member 120 fails, thereby separating arms 116 of anchor extension 114 from anchor body 106. Bending of arms 116 of anchor extension 114 in such a fashion can disturb tissue and other anatomical features proximal to bone anchor 100. Nonetheless, some surgical personnel might desire to separate arms 116 of anchor extension 114 from anchor body 106 by repeatedly bending arms 116 of anchor extension 114. To accommodate judgment of surgical personnel pertaining to which method of separation given situations might indicate, some embodiments include separation members 120 adapted to allow surgical personnel a choice between separating anchor extension 114 from anchor body 106 by bending, torquing, or a combination thereof.

In some embodiments, spinal stabilization rod 24 can be reduced into one or more bone anchors 100 and other bone anchors 100 can be awaiting reduction of spinal stabilization rod 24 into themselves. Because of deformities and other conditions affecting spine 10, these particular bone anchors may be oriented in directions pointing substantially away from spinal stabilization rod 24 in partially unreduced, intermediary positions. In such cases, surgical personnel can grasp spinal stabilization rod 24 and force spinal stabilization rod 24 to the vicinity of threaded slot 118 of anchor extension 114 of a particular bone anchor 100. Forcing spinal stabilization rod 24 into such positions may temporarily cure or correct the deformities or other conditions affecting spine 10. By reducing spinal stabilization rod 24 into the remaining bone anchors 100, it can be the case that spinal stabilization system 22 permanently corrects the deformity or conditions involved. In some embodiments, though, partially reduced spinal stabilization rod 24 might lie within threaded slot 118 of anchor extension 114 or within threaded slot 110 of anchor body 106 of one, or more, bone anchors 100. In such cases, spinal stabilization rod 24 can be reduced into other bone anchors as desired without forcing spinal stabilization to these particular bone anchors. Thus, when desired, additional bone anchors 100 can be used to reduce spinal stabilization rod 24. Thus spinal stabilization systems 22 (see FIG. 2) can be implanted in patients to stabilize spine 10, to treat various diseases thereof, to correct deformities thereof etc. by various methods.

In the foregoing specification, specific embodiments have been described with reference to the accompanying drawings. However, as one skilled in the art can appreciate, embodiments of the bone anchor disclosed herein can be modified or otherwise implemented in many ways without departing from the spirit and scope of the disclosure. Accordingly, this description is to be construed as illustrative only and is for the purpose of teaching those skilled in the art the manner of making and using embodiments of a bone anchor. It is to be understood that the embodiments shown and described herein are to be taken as exemplary. Equivalent elements or materials may be substituted for those illustrated and described herein. Moreover, certain features of the disclosure may be utilized independently of the use of other features, all as would be apparent to one skilled in the art after having the benefit of this description of the disclosure.

1. A collar for reducing a spinal stabilization rod in conjunction with a bone fastener for attaching the collar to a boney structure, the collar comprising:
a body including a first pair of arms defining a first threaded slot to accept the spinal stabilization rod and to accept a closure member;
an extension of the body including a second pair of arms corresponding to the first pair of arms and defining a second threaded slot corresponding to the first threaded slot; and
a separation member coupling the body and the extension, wherein when the spinal stabilization rod is reduced through the extension of the separation member, a tensile force arises from the reduction of the spinal stabilization rod through the extension and wherein when the selected torque is applied to the extension, the separation member transmits the selected torque from the extension to the body
and wherein the separation member yields in torsion when more than the selected torque is applied to the extension.

2. The collar of claim 1 wherein the first pair of arms define a first peripheral length, wherein the separation member defines a second peripheral length, and wherein the second peripheral length is substantially less than the first peripheral length.

3. The collar of claim 1 wherein the separation member is positioned adjacent to the threaded slots.

4. The collar of claim 1 further comprising a tensile force relief feature proximal to the separation member.

5. The collar of claim 1 wherein at least one of the body or the extension defines a corner proximal to the separation member.

6. The collar of claim 1 further comprising an anti-splay feature of the second threaded slot wherein the anti-splay feature mitigates a moment arising from the reduction of the spinal stabilization rod through the extension.

7. The collar of claim 1 wherein the tensile force is at most about 1000 pounds.

8. The collar of claim 1 wherein the selected torque is about 8.3 foot-pounds.

9. The collar of claim 1 wherein the separation member is a webbing.

10. The collar of claim 1 wherein the separation member further defines a gap between the threads of the first and second threaded slots.

11. A method of reducing a spinal stabilization rod comprising:
attaching a collar to a boney structure with a bone fastener, the collar including a body including a first of pair of arms defining a first threaded slot, an extension of the body including a second pair of arms corresponding to the first pair of arms and defining a second threaded slot corresponding to the first threaded slot, and a separation member coupling the body and the extension;
reducing the spinal stabilization rod through the extension of the collar; and
separating the extension from the body by applying a selected torque to the extension wherein the member yields in torsion.

12. The method of claim 11 wherein the extension is separated from the body by one substantially continuous motion, thereby leaving adjacent tissues substantially undisturbed.

13. The method of claim 11 wherein the reduction of the spinal stabilization rod further mitigates a moment arising from the reduction of the spinal stabilization rod.

14. The method of claim 11 wherein the separation member transmits a tensile force arising from the reduction of the spinal stabilization rod between the body and the extension.

15. A collar for reducing a spinal stabilization rod into a bone fastener, the collar comprising:
a body including a first pair of arms defining a first peripheral length and a first threaded slot to accept the spinal stabilization rod and to accept a closure member;
an extension of the body including a second pair of arms corresponding to the first pair of arms and defining a second threaded slot corresponding to the first threaded slot;
a separation member coupling the body and the extension, defining a second peripheral length and a gap between the threads of the first and second threaded slots, and being positioned adjacent to the threaded slots, wherein the second peripheral length is substantially less than the first peripheral length, wherein the separation member transmits up to about a selected torque to the body when the selected torque is applied to the extension, and wherein the member yields in torsion when more than about the selected torque is applied to the extension;
a tensile relief feature proximal to the separation member; and
an anti-splay feature of the second threaded slot, wherein the anti-splay feature mitigates a moment arising from the reduction of the spinal stabilization rod through the extension.
